(19) United States
(12) Patent Application Publication
(10) Pub. No.: US 2003/0206945 A1
(43) Pub. Date: Nov. 6, 2003

(54) Sticking plaster for controlled release of natural interferon

(76) Inventors: Giulio Tarro, Roma (IT); Renzo Brozzo, Roma (IT)

Correspondence Address: GRIFFIN & SZIPL, PC, SUITE PH-1, 2300 NINTH STREET, SOUTH, ARLINGTON, VA, 22204, US

(21) Appl. No.: 10/418,172

(22) Filed: Apr. 18, 2003

Related U.S. Application Data
(63) Continuation of application No. 09/720,027, filed on Dec. 20, 2000, now abandoned.
Application No. 09/720,027 is a 371 of international application No. PCT/EP98/03809, filed on Jun. 22, 1998.

(51) Int. Cl. A61K 38/21; A61K 9/70

(52) U.S. Cl. 424/449; 424/85.7

(57) ABSTRACT

A sticking plaster for controlled release of natural human α-interferon for use in viral therapies, in particular anti-herpes therapies, comprises a thin flexible support and a layered adhesive forming a matrix, a therapeutically effective quantity of the interferon being dispersed in said matrix.

### BACKGROUND OF THE INVENTION

The invention relates to a sticking plaster for controlled release of natural interferon for treating viral infections, in particular herpes infections. More particularly, the invention concerns an adhesive plaster for the controlled release of natural human α-interferon (nHuαIFN) derived from leucocytic or lymphoblastoid cells, for use in therapies designed to counter lip and genital herpes infections (caused by the viruses HSV-1 and HSV-2), Herpes Zoster (HZV), Zoster's neuritis (HZV) and flat verrucas (HPV).

For some time, nHuαIFN has been used in studies in the field of viral infections and has found a clearly-defined role in the last few years in therapies against acute and chronic viral pathologies, where it is often considered to be a “first choice drug”.

The majority of the mechanism by which nHuαIFN acts is known depends on its direct interaction with the target cells and/or on the inducement of a biological response in the host organism.

The activity of the α-interferon as an immunomodulant plays an important role at the organic level in the defence mechanism against viral infections. nHuαIFN is administered parenterally (intravenously, intramuscularly, subcutaneously), orally and topically (ointments and eye-washes). The dosages usually recommended are in the order of millions of I.U. (International Units) for parenteral administrations, hundreds of thousands of I.U. for topical preparations and hundreds of I.U. for oral administrations. These administrations (excluding oral administrations) are prejudiced by a series of more or less serious drawbacks, which make interferon therapy unattractive to patients.

The most remarkable disturbances range from serious collateral effects, such as nausea, vomiting or obnubilation of the senses, which require the interruption of the therapy, to certain less significant ones, such as the troublesome repetition of the administrations and the greasiness of the ointments.

In addition, as a result of the rapid fall-off in the plasmatic concentrations of the active ingredient, it is sometimes necessary to resort to overdosage in order to achieve satisfactory pharmacological effects, running the risk of generating serious collateral effects and high therapy costs (the therapy is an assisted one practised with hospital or day hospital care).

There is therefore a need for a formulation designed to cope with the above-mentioned pathologies when diagnosed with certainty, which would enable the dose of nHuαIFN to be reduced, without altering its therapeutic effectiveness, and which could be used with a more pleasant method of administration and have stronger, more lasting pharmacological effects.

The authors of this invention have developed a sticking plaster for controlled release of nHuαIFN that overcomes the inconveniences of the known formulations.

### SUMMARY OF THE INVENTION

In fact, the application of an occlusive medication and a system of controlled release enables very small doses of nHuαIFN to be administered in the very place where the viral lesion is situated.

The subject matter of the invention consists of a sticking plaster for the controlled release of natural human α-interferon for local transcutaneous absorption comprising:

a thin, inert, flexible support comprising an inner side and an outer side;

a layered adhesive on the inner side of said support so as to form a matrix;

a therapeutically effective quantity of said interferon dispersed in the matrix;

in which said support is not repellent for said matrix, but is repellent for the interferon.

The support is preferably on a polyurethane base. The adhesive preferably comprises substances on an acrylic base.

The quantities of nHuαIFN present are preferably from 150 I.U. (International Units) to 150,000 I.U., more preferably from 150 I.U. to 1,500 I.U., 15,000 I.U. or 150,000 I.U., which in the doctors's opinion would enable the dosage to be varied. The sticking plaster normally has an applicatoin autonomy of 24 hours.

The sticking plaster should preferably comprise a release strip, as an element of protection of the inner side, which can be removed at the moment when it is applied, substantially of the same dimensions as the support and preferably manufactured with a central pre-cut.

It would be an advantage that the sticking plaster be available in various shapes, to facilitate its use in practice: a square shape and anatomic shapes for specific parts of the body.

### PREFERRED EMBODIMENT

The invention will now be described for the purposes of illustration but not limitation, with reference to the method used to prepare a 3×3 cm sticking plaster. A sticking plaster for transcutaneous absorption, for controlled release of nHuαIFN derived from leucocytic or lymphoblastoid cells, is prepared as follows: on a plastic support with a square shape and rounded corners, measuring 3×3 cm, a matrix is spread that consists of: an acrylic base, acrylic acid, ethylenic glycol, a water alcohol solution and nHuαIFN of the type and dosage desired, which in its turn is covered with a removable protective layer (release strip) manufactured with a central pre-cut.

Although this invention has been described for the purposes of illustration but not limitation, according to the preferable forms in which it should be produced, it should be understood that variations and/or modifications may be made by experts in the field, without for this reason exiting from the scope of protection.

1. A sticking plaster for the controlled release of natural human α-interferon for transcutaneous absorption, comprising:
a thin, flexible support, comprising an inner side and an outer side;
a layered adhesive on the inner side of said support, so as to form a matrix;
a therapeutically effective quantity of said interferon dispersed in said matrix;
in which said support is not repellent for said matrix, but is repellent for the interferon.

2. A sticking plaster according to claim 1 in which said support is on a base of polyurethanes.

3. A sticking plaster according to claim 1 or 2 in which said adhesive is on an acrylic base.

4. A sticking plaster according to one of the preceding claims in which said interferon is contained in quantities ranging from 150 I.U. to 150,000 I.U.

5. A sticking plaster according to one of the preceding claims in which said interferon is contained in quantities of 150 I.U., 1,500 I.U., 15,000 I.U. or 150,000 I.U.

6. A sticking plaster according to one of the preceding claims further comprising a removable protective element for the inner side of said support, of substantially the same dimensions as the support.

7. The use of natural human α-interferon for preparing a sticking plaster for the controlled release of interferon for transcutaneous absorption, for use in anti-herpes therapies.
